(19) United States
(12) Patent Application Publication
(10) Pub. No.: US 2024/0373871 A1
(43) Pub. Date: Nov. 14, 2024

(54) Treated Soybean Oil

(71) Applicant: General Mills, Inc., Minneapolis, MN (US)

(72) Inventors: Catherine Davlin, Minneapolis, MN (US); Amelia Elizabeth Frazier, Coon Rapids, MN (US); Jon Duke Seibold, Mahtomedi, MN (US); Harold Jack Stevens, Delano, MN (US)

(73) Assignee: General Mills, Inc., Minneapolis, MN (US)

(21) Appl. No.: 18/686,584

(22) PCT Filed: Aug. 9, 2022
(86) PCT No.: PCT/US2022/039772

§ 371 Date: Feb. 26, 2024

Related U.S. Application Data
(60) Provisional application No. 63/243,345, filed on Sep. 13, 2021.

(51) Int. Cl. A23D 9/04; A23L 5/20; A23L 5/49; A23L 7/122; C07C 45/41

(52) U.S. Cl. CPC A23D 9/04; A23L 5/21; A23L 5/273; A23L 5/49; A23L 7/122; C07C 45/41

(57) ABSTRACT

Methods of treating edible oils, such as soybean oil, to reduce furan fatty acid content to produce edible oils that are more resistant to off-flavor development when used in food applications are described. Some methods include heat treatment of an edible oil in the presence of oxygen followed by deodorization to produce a heat-treated oil. Some methods includes treating an edible oil under conditions that promote conversion of furan fatty acids to 3-methyl-2,4-nonanedione (3MND) and/or 3-hydroxy-3MND (3H-3MND) in the presence of activated carbon to produce a carbon-treated oil. Also described are treated edible oils and foods that include treated edible oils.

### BACKGROUND

Fats and oils are used in many foods, including commercially available foods, such as ready-to-eat (RTE) breakfast cereals, snacks, granola, cookies and other baked goods, and the like. Many vegetable oils, however, are prone to oxidation over shelf life, contributing to off-flavors. Although partially hydrogenated oils provide functionality and stability against off flavors over shelf life, partially hydrogenated oils also contain high levels of trans fatty acids that were found to be detrimental to human health when consumed. As one way to improve shelf life of vegetable oils, and also avoid trans fatty acids, some canola, soybean, sunflower and safflower varieties were bred to have high oleic acid content. High-oleic canola, soybean, sunflower, and safflower oils have become increasingly popular for use in commercially available foods due to their increased resistance to off flavors over shelf life.

### SUMMARY

The present disclosure relates to methods of treating edible vegetable oils to produce treated oils that have resistance to off-flavors over shelf life.

Methods of making treated soybean oils having reduced furan fatty acids content are provided herein. In some embodiments, a method is a method of making a carbon-treated soybean oil. A method of making a carbon-treated soybean oil can include providing a soybean oil; treating the soybean oil under conditions that promote conversion of furan fatty acids to 3MND in the presence of an amount of activated carbon to produce a carbon-treated soybean oil; and collecting the carbon-treated soybean oil, wherein the carbon-treated soybean oil has a reduced furan fatty acids content relative to the soybean oil. In some embodiments, conditions that promote conversion of furan fatty acids to 3MND can include maintaining the soybean oil at a temperature of about 90° F. (32.2° C.) to about 120° F. (48.9° C.) in the presence of oxygen. In some embodiments, conditions that promote conversion of furan fatty acids to 3MND can include oil movement. In some embodiments of a method of making a carbon-treated soybean oil, the amount of activated carbon can be at least 6% by weight of the soybean oil. In some embodiments of a method of making a carbon-treated soybean oil, the carbon-treated soybean oil can have a furan fatty acids content is less than 100 mg/kg. In some embodiments of a method of making a carbon-treated soybean oil, at least a portion of the soybean oil can be a high oleic soybean oil. In some embodiments of a method of making a carbon-treated soybean oil, at least a portion of the soybean oil can be bleached and/or deodorized. A carbon-treated soybean oil made according to a method of making a carbon-treated soybean oil provided herein is also provided.

In some embodiments, a method is a method of making a deodorized, heat-treated soybean oil. A method of making a deodorized, heat-treated soybean oil can include providing a soybean oil; maintaining the soybean oil at a temperature of 170° F. (76.7° C.) to 460° F. (237.8° C.) in the presence of oxygen for at least 30 minutes to make a heat-treated soybean oil; deodorizing the heat-treated soybean oil to produce a deodorized, heat-treated soybean oil; and collecting the deodorized, heat-treated soybean oil, wherein the deodorized, heat-treated soybean oil has a reduced furan fatty acid content relative to the soybean oil. In some embodiments, the maintaining step can be performed at a temperature of 260° F. (126.7° C.) to 300° F. (148.9° C.) for about 1 hour to about 6 hours. In some embodiments, the maintaining step can be performed in the presence of a bleaching clay. In some embodiments, the maintaining step can be performed in the presence of activated carbon. In some embodiments, at least a portion of the soybean oil can be a high oleic soybean oil.

In some embodiments of a method of making a deodorized, heat-treated soybean oil, additional steps can be included. Such steps can include treating the deodorized, heat-treated soybean oil under conditions that promote conversion of furan fatty acids to 3MND in the presence of an amount of activated carbon to produce a carbon-treated soybean oil; and collecting the carbon-treated soybean oil. In some embodiments, conditions that promote conversion of furan fatty acids to 3MND can include maintaining the soybean oil at a temperature of about 90° F. (32.2° C.) to about 120° F. (48.9° C.) in the presence of oxygen. In some embodiments, conditions that promote conversion of furan fatty acids to 3MND can include oil movement. In some embodiments of a method of making a carbon-treated soybean oil, the amount of activated carbon can be at least 6% by weight of the soybean oil.

A method of reducing furan fatty acid content in an oil, is also provided herein. The method can include providing an untreated oil having a furan fatty acid content of at least 100 mg/kg; carbon-treating and/or heat-treating the untreated oil to produce a treated oil, where: carbon-treating the oil comprises treating the oil under conditions that promote conversion of furan fatty acids to 3MND in the presence of an amount of activated carbon and heat-treating the oil comprises maintaining the oil at a temperature of 170° F. (76.7° C.) to 460° F. (237.8° C.) in the presence of oxygen for at least 30 minutes; and collecting the treated oil, wherein the treated oil has a reduced furan fatty acids content relative to the untreated oil. In some embodiments, the treated oil can have a furan fatty acid content that is reduced by at least 15%, at least 20%, or at least 25% relative to the untreated oil. In some embodiments, the treated oil can have a fatty acid content that is less than 100 mg/kg.

A carbon-treated soybean oil having a furan fatty acids content of less than 100 mg/kg is also provided herein. In some embodiments, at least a portion of the carbon-treated soybean oil can be a high oleic soybean oil. In some embodiments, at least a portion of the carbon-treated soybean oil can be bleached or deodorized.

A food comprising a carbon-treated soybean oil provided herein is also provided.

Also provided is a method of making a grain-based food product, including combining ingredients to form an uncooked composition, where the uncooked composition includes a soybean oil having a furan fatty acids content of at least 100 mg/kg, and substantially no reducing sugar and a pH of less than 6; and cooking the uncooked composition to produce the grain-based food product. In some embodiments, the grain-based food product can be a granola or a granola bar.

Also provided is a method of making a grain-based food product, providing a grain-based food piece; and applying a coating to a surface of the grain-based food piece to make the grain-based food product, where the coating includes soybean oil having a furan fatty acids content of at least 100 mg/kg, and no reducing sugar and a pH of less than 6. In some embodiments, the grain-based food product can be a breakfast cereal.

These and various other features and advantages will be apparent from a reading of the following detailed description.

### DETAILED DESCRIPTION

Edible vegetable oils, such as soybean oil, canola oil, sunflower, and safflower oil can suffer from the development of off-flavors over shelf life due to relatively high content of fatty acids that are susceptible to oxidation, such as linoleic acid and linolenic acid. To improve shelf life, some soybean, canola, sunflower, and safflower varieties were bred to have naturally increased oleic acid content, which is more resistant to oxidation. High-oleic vegetable oils have been increasingly used in shelf stable foods because of their stability over shelf life and during frying. High-oleic canola (HOC) oil has been particularly popular for use because it offers neutral flavor, as well as oxidative stability. However, because of such high demand, HOC oil can sometimes be limited in supply and/or relatively expensive.

Although soybean oil isn't as neutral in flavor as canola oil, in many applications, it was initially believed that a refined, bleached, and deodorized (RBD) high-oleic soybean (HOSoy) oil could be substituted for RBD HOC oil without significantly impacting flavor, especially since RBD HOSoy oil is much more oxidatively stable than RBD commodity soybean oil, and is neutral in flavor. Surprisingly, however, it was discovered that RBD HOSoy oil could not be substituted for RBD HOC oil in some foods that include components that participate Maillard browning. Such foods, such as RTE breakfast cereal pieces, granola, and grain-based snacks, developed a noticeable off-flavor, described as green, painty, beany, and/or minty, often almost immediately after baking or toasting, but also sometimes developed an off-flavor over shelf life in some products where the RBD HOSoy oil was not subjected to baking or toasting (e.g., as an oil-based coating). In addition, such foods normally had a desirable flavor associated with Maillard browning (“brown, sweet flavor”) when made with RBD HOC oil, but lacked the brown, sweet flavor when made with RBD HOSoy oil.

In an effort to reduce or eliminate the off-flavor in foods made with HOSoy oil, it was discovered and is disclosed herein, that edible oils that include furan fatty acids (FuFA) at levels of at least 100 mg/kg, such as soybean oil and HOSoy oil, can be treated using particular methods that include using, in the presence of oxygen, activated carbon and/or heat to reduce or eliminate off-flavor. Surprisingly, methods that include treatment with activated carbon also at least partially, and sometimes fully, restore a brown, sweet flavor associated with Maillard browning.

It was previously reported that FuFA in soybean oil was oxidized by light to produce 3-methyl-2,4-nonanedione (3MND) and/or 3-hydroxy-3MND (3H-3MND), which were associated with off-flavors in soybean oil exposed to light (Sano, et al., J. Agric. Food Chem. 2017, 65, 2136-2140). While conversion of FuFA to 3MND and/or 3H-3MND during Maillard browning was not previously identified, it was initially hypothesized that the off-flavor discovered above in foods made with HOSoy due to the presence of 3MND and/or 3H-3MND being formed during Maillard browning during baking or toasting of such foods, or during shelf life, or due to 3MND and/or 3H-3MND interacting with Maillard browning products to produce an off-flavor. Thus, it was initially hypothesized that foods made using oils treated using methods disclosed below had reduced levels 3-methyl-2,4-nonanedione (3MND) and/or 3-hydroxy-3MND (3H-3MND), which was hypothesized to reduce off-flavor. Unexpectedly, however, the beneficial effects of the disclosed methods on a treated edible oil appear to be independent of 3MND and 3H-3MND levels (either alone or in combination) in products made using a treated edible oil.

A method of making a carbon-treated oil is provided herein. Any edible oil having a FuFA content of at least 100 mg/kg, such as commodity soybean oil or HOSoy oil, can be used in a method of making a carbon-treated oil. A suitable edible oil is typically refined, but can also be optionally bleached (RB) or bleached and deodorized (RBD). In some cases, an edible oil suitable for use in a method of making a carbon-treated oil can be heat-treated according to a method described herein, such that the oil is both heat-treated and carbon-treated.

A method of making a carbon-treated oil includes treating a suitable oil, in the presence of an amount of activated carbon, under conditions that promote conversion of FuFA to 3MND to produce the carbon-treated oil. As used herein, conditions that promote conversion of FuFA to 3MND include maintaining an oil being treated at a temperature of about 90° F. (32.2° C.) to about 120° F. (48.9° C.) in the presence of oxygen (e.g., in the presence of an atmosphere with an oxygen content of at least 5%, at least 10%, from about 15% to about 30%, or about 18% to about 22%). In some embodiments, conditions that promote conversion of FuFA to 3MND can include incorporation of oil movement (e.g., agitation, flow, sparging, and/or circulation). Oil movement can increase reaction rate as well as more evenly expose the oil to activated carbon and/or oxygen. Without being bound by theory, it is believed that a method of making a carbon-treated oil described herein reduces FuFA content in a treated oil by converting at least a portion of FuFA to 3MND that can be adsorbed by activated carbon. It is further believed that although 3MND levels in foods made with a carbon-treated oil do not appear to correlate with development of off-flavor, 3MND carbon adsorption from oil being treated does appear to correlate with whether the resulting carbon-treated oil will produce a good tasting food. See, the Examples provided herein.

Any activated carbon that is safe for use in food can be used in a method of making a carbon-treated oil. For example, an activated carbon can be in granulated form or powder form, and can be wood-based or coal-based.

An amount of activated carbon used in carbon treatment can be adjusted based on, for example, the type of process used (e.g., batch process or continuous process), the amount of FuFA in the oil, the time the oil is exposed to activated carbon, the efficiency of the particular activated carbon at adsorbing 3MND, or the cost of the activated carbon being used. For example, in a batch process, to provide a good balance of effectiveness of carbon treatment and cost of activated carbon, in some embodiments, an amount of activated carbon used can be from about 1% to about 15% (e.g., about 3% to about 10%, or at least 6%) by weight of oil being treated. In another example, an amount of activated carbon can be adjusted for use in a continuous process to account for oil volume, flow rate, and the like. In another example, a powdered activated carbon can be used at a lower rate (e.g., about 1% to about 8% by weight of oil in a batch process), while a granulated carbon might be used at a higher rate (e.g., about 5% to about 15% by weight of oil in a batch process).

An oil can be treated in the presence of activated carbon under conditions that promote conversion of FuFA to 3MND for sufficient time to result in a reduction in FuFA content in the oil. For example, FuFA content in a carbon-treated oil can be reduced by at least 15% (e.g, at least 20%, or at least 25%) relative to FuFA content prior to carbon treatment. In some embodiments, FuFA content in a carbon-treated oil provided herein can be less than 100 mg/kg (e.g., less than 90 mg/kg, less than 80 mg/kg, or less than 75 mg/kg).

The amount of time sufficient to achieve the desired FuFA content reduction can be affected based on, for example, the conditions used, the efficiency of the particular activated carbon at adsorbing 3MND, the process used (e.g., continuous or batch process), and the amount of activated carbon used relative to the amount of oil being process. Generally, a higher the temperature used to promote conversion of FuFA to 3MND can require less time in the presence of activated carbon to achieve sufficient reduction in FuFA content. Similarly, use of more activated carbon, a higher efficiency activated carbon, and/or an activated carbon with greater surface area (e.g., powdered activated carbon), can require less time to achieve sufficient reduction in FuFA content.

In some embodiments, time sufficient to result in a reduction in FuFA content in an oil can be at least 20 minutes (e.g., at least 30 minutes, at least 45 minutes, at least 1 hour). For example, in some embodiments, an oil can be treated in the presence of powdered activated carbon for about 45 minutes to about 90 minutes (e.g., about an hour) under conditions that promote conversion of FuFA to 3MND, where the conditions include maintaining the oil at about 100° F. to about 115° F. (e.g., about 110° F.) in the presence of oxygen with agitation or circulation of the oil, to achieve a FuFA content reduction of about 20% to about 40% relative to the FuFA content prior to carbon treatment.

It is to be understood that an oil may be treated in the presence of activated carbon for a very long time (e.g., several hours, days, or weeks) before being collected. However, it may be practical to limit the exposure for the purposes of efficiency and cost. For example, in some embodiments, an oil can be treated in the presence of activated carbon for 30 minutes to 4 hours (e.g., 45 minutes to 2 hours, or about an hour) for the sake of cost and/or efficiency, even though the method is still effective if the oil is in contact with activated carbon for a longer period of time.

Following carbon treatment, a carbon-treated oil can be collected by any appropriate method and/or equipment. Generally, a carbon-treated oil is collected by collecting oil that is not adsorbed by the activated carbon (e.g., oil not requiring extraction from the activated carbon). A carbon-treated oil provided herein can have a FuFA content of less than 100 mg/kg (e.g., 90 mg/kg or less, 80 mg/kg or less, or 75 mg/kg or less). In some embodiments, can impart a brown, sweet flavor in a food (e.g., a grain-based food) made with the carbon-treated oil.

A method of making a heat-treated oil is also provided herein. Any edible oil having a FuFA content of at least 100 mg/kg, such as commodity soybean oil or HOSoy oil, can be used in a method of making a heat-treated oil. A suitable edible oil is typically refined, but can also be optionally bleached (RB) or bleached and deodorized (RBD). In some embodiments, a method of making a heat-treated oil may be beneficially incorporated into a bleaching process prior to deodorization.

A method of making a heat-treated oil includes maintaining a suitable oil at a temperature of 170° F. (76.7° C.) to 460° F. (237.8° C.) (e.g., 260° F. to 300° F.) in the presence of oxygen for at least 30 minutes (e.g., 30 minutes to about 6 hours, or about 1 hour to about 6 hours) to make a heat-treated oil. The amount of time used can be adjusted based on the temperature at which an oil is maintained in a method of making a heat-treated oil. For example, a method of making a heat-treated oil that includes maintaining an oil at a temperature of 260° F. (126.7° C.) to 300° F. (148.9° C.) can be performed for about 1 hour to about 6 hours (e.g., about 2 hours to about 4 hours), while a method that includes maintaining an oil at a temperature of greater than 300° F. may be performed for less than 2 hours, and a method that includes maintaining an oil at less than 260° F. may be performed for at least 3 hours.

In some embodiments, a method of making a heat-treated oil can be performed in the presence of a bleaching clay and/or activated carbon as part of a bleaching process. However, unlike a typical bleaching process performed on edible oils, which is performed under a vacuum with little oxygen (e.g., less than 1% oxygen) present in the atmosphere contacting the oil, a bleaching process used as part of making a heat-treated oil described herein is performed in the presence of oxygen. For example, a heat treatment described herein can be performed in the presence of an atmosphere with an oxygen level of greater than 5% (e.g., at least 10%, about 15% to about 30%, or about 18% to about 22%). In some embodiments, an oil can be circulated, agitated, sparged, or the like to increase contact with oxygen. Without being bound by theory, it is believed that a method of making a heat-treated oil described herein promotes oxidative breakdown of FuFA in an oil to result in reduced FuFA content in the oil. A heat-treated oil provided herein can have a FuFA content that is reduced by at least 15% (e.g, at least 30%, at least 40%, or at least 50%) relative to FuFA content prior to heat treatment. In some embodiments, FuFA content in a heat-treated oil provided herein can be less than 100 mg/kg (e.g., less than 90 mg/kg, less than 75 mg/kg, or less than 60 mg/kg).

Following heat treatment, it is preferred that a heat-treated oil be deodorized to produce a deodorized, heat-treated oil. Deodorization of a heat-treated oil can be done using any known oil deodorization process. Following deodorization, a deodorized, heat-treated oil can be collected by any appropriate method and/or equipment. In some embodiments, a deodorized, heat-treated oil provided herein can have a FuFA content of less than 100 mg/kg (e.g., less than 90 mg/kg, less than 75 mg/kg, or less than 60 mg/kg).

In some embodiments, a heat-treated oil can be subjected, before or after deodorization, to carbon treatment as described above. In some embodiments, an oil that is both carbon-treated and heat-treated can impart a brown, sweet flavor in a food (e.g., a grain-based food) made with the carbon-treated and heat-treated oil.

Also provided herein are food products containing a heat-treated and/or carbon-treated oil provided herein. It is particularly beneficial to use a heat-treated and/or carbon-treated oil provided herein in producing grain-based food products (e.g., RTE breakfast cereal, granola, cookies, and the like). That is, because off-flavors from untreated oils that contain FuFA levels above 100 mg/kg are particularly noticeable in grain-based foods either immediately or over shelf life, heat-treated and/or carbon-treated oil provided herein can improve flavor and/or enjoyable shelf life of such grain-based foods. For example, using a heat-treated and/or carbon-treated oil in a grain-based food that is baked and/or toasted while the oil is present, such as a granola, a granola bar, or a baked grain-based snack, can improve the flavor of the grain-based snack. In another example, using a heat-treated and/or carbon-treated oil as a topical application on a grain-based food that is not baked and/or toasted following application of the oil can reduce off-flavor development over shelf life.

In some embodiments, a food product containing a heat-treated and/or carbon-treated oil can be uncooked, such as a dough (e.g., for cookies, bread, or the like), a batter (e.g., for brownies, pancakes, muffins, or the like) or a dry mix (e.g., for cookies, crackers, biscuits, cakes, or the like) intended to be cooked (e.g., baked or toasted) by a consumer or a commercial kitchen. Such an uncooked food product can benefit from inclusion of a heat-treated and/or carbon-treated oil by having improved flavor upon cooking and/or over shelf life.

It is to be understood that, while a benefit of heat-treated and/or carbon-treated oil may be more readily apparent in producing grain-based foods, other foods, such as baked and/or toasted dairy-based snacks (e.g., cheese-based crackers), protein-based foods, and the like, may also benefit from the use of a heat-treated and/or carbon-treated oil.

Also provided herein are methods of making a grain-based food product (e.g., a breakfast cereal, granola, cookies, or a granola bar) having at least some of the benefits described above with respect to use of a heat-treated and/or carbon-treated oil, without requiring heat treatment and/or carbon treatment of an oil having a FuFA content of at least 100 mg/kg. Such a method includes reducing or eliminating Maillard reactions within the food, despite it being typically desirable in grain-based food products for a Maillard reaction to occur to develop desirable flavors. Without being bound by theory, it is believed that off-flavor in a grain-based food containing an oil with at least 100 mg/kg FuFA is amplified by a Maillard reaction during cooking or during shelf life. In some embodiments, a method of making a grain-based food product can include combining ingredients to form an uncooked composition (e.g., a cookied dough or an unbaked granola), where the uncooked composition includes an oil having a FuFA content of at least 100 mg/kg (e.g., a soybean oil that has not been heat-treated and/or carbon-treated in a method described above) and where the uncooked composition includes substantially no reducing sugar (e.g., less than 0.5%, less than 0.1%, or 0%), and cooking the uncooked composition to produce a grain-based food product. In some embodiments, a method of making a grain-based food product can include combining ingredients to form an uncooked composition, where the uncooked composition includes an oil having a FuFA content of at least 100 mg/kg (e.g., a soybean oil that has not been heat-treated and/or carbon-treated in a method described above), where the uncooked composition includes substantially no reducing sugar (e.g., less than 0.5%, less than 0.1%, or 0%), and where the uncooked composition has a pH of less than 6 (e.g., less than 5.5), and cooking the uncooked composition to produce a grain-based food product.

In some embodiments, a method of making a grain-based food product can include providing a grain-based food piece (e.g., a RTE breakfast cereal piece, or a snack piece, and applying a coating to a surface of the grain-based food piece to make the grain-based food product, where the coating includes an oil having a FuFA content of at least 100 mg/kg (e.g., a soybean oil that has not been heat-treated and/or carbon-treated in a method described above) and where the coating includes no reducing sugar (i.e., 0%). In some embodiments, a method of making a grain-based food product can include providing a grain-based food piece (e.g., a RTE breakfast cereal piece, or a snack piece, and applying a coating to a surface of the grain-based food piece to make the grain-based food product, where the coating includes an oil having a FuFA content of at least 100 mg/kg (e.g., a soybean oil that has not been heat-treated and/or carbon-treated in a method described above), where the coating includes no reducing sugar, and where the coating has a pH of less than 6 (e.g., less than 5.5).

The following examples provide additional support to the above-described invention, and are not meant to limit the scope of the invention.

### EXAMPLES

### Example 1—Carbon Treatment

HOSoy oil (Samples 1, 2, and 4-11) and commodity soybean oil (Sample 3 was carbon-treated by maintaining the oil at various temperatures and times with agitation in the presence of oxygen and a wood-based activated carbon at a rate of 6-8% by weight of the oil treated. Table 1 shows the conditions tested and whether off-flavor was detected in granola made with the treated oil.

TABLE 1

| | | | | Rate of | |
|---|---|---|---|---|---|
| | Temperature | | | activated | Off-flavor |
| Sample | (° F.) | Time | | carbon | detection |
| Control | NA | NA | | NA | Yes |
| (untreated) | | | | | |
| 1 | 110 | 1 | hour | 8% | No |
| 2 | 110 | 1 | hour | 6% | No |
| 3 | 110 | 1 | hour | 8% | No |
| 4 | 110 | 5 | minutes | 1.5% | Yes |
| 5 | 110 | 5 | minutes | 6% | Yes |
| 6 | 110 | 1 | hour | 0.5% | Yes |
| 7 | 110 | 1 | hour | 1.5% | Yes |
| 8 | 110 | 1 | hour | 2% | Yes |
| 9 | 110 | 1 | hour | 4% | Yes |
| 10 | 110 | 1 | hour | 7% | No |
| 11 | Room | 1 | hour | 8% | Yes |

Temperature

As can be seen in Table 1, conditions that include a temperature of 110° F. (43.3° C.) and a time of 1 hour in the presence of greater than 4% carbon by weight of oil resulted in little to no off-flavor detected. These results suggest that temperatures and times sufficient to oxidize FuFA are necessary to achieve off-flavor reduction.

The results above could be repeated with several different types of activated carbon, with varying degrees of success. Generally, the tested wood-based activated carbon was more effective at reducing off-flavor than the tested coal-based activated carbon and the tested powdered activated carbon was more effective at reducing off-flavor than the tested granular activated carbon. A tested coconut-based granulated activated carbon was not effective. It is believed that the tested wood-based activated carbon was more effective at adsorbing 3MND and/or 3H-3MND due to having a more open pore structure than the tested coal-based activated carbon and coconut-based activated carbon, while the tested powdered activated carbon is more efficient due to having a larger surface area relative to granular activated carbon that was tested. However, as discussed below, 3MND and 3H-3MND levels in granola surprisingly appeared to be at least somewhat independent of whether the granola exhibited an off-flavor.

To determine the effect of carbon treatment on FuFA content, 3MND content, and 3H-3MND content, levels of these molecules were measured using gas chromatography-mass spectrophotometry (GC-MS) in untreated oil, carbon-treated oil (1 hour at 110° F. with wood-based activated carbon), extracts from activated carbon used to make carbon-treated oil, and oil extracted from granola made using each oil. Briefly, granola was made by combining rolled oats with a sugar slurry that contains about 54% of the appropriate oil, then baking for approximately 40 minutes at 300° F. Extracts from activated carbon were performed on duplicate samples of activated carbon used for treatment of each of the indicated samples. One of the duplicate samples was extracted using a polar solvent (methanol), and the other of the duplicate samples was extracted using a non-polar solvent (hexane). Briefly, extraction was performed by combining samples with the selected solvent and vigorously agitated, and the solvent decanted into an evaporative flask. The extraction was performed twice more for each sample, and the pooled collected solvent was evaporated down to near dryness to produce samples for GC-MS testing. Oil was extracted from granola using the following protocol. Samples were prepared prior to extraction by placing a 60 g sample into a blender glass jar equipped with a bladed cap. The sealed jar was wrapped with aluminum foil and frozen at −60° C. for 1 hour. The frozen sample was ground as follows: blend for 30 seconds, shake 1 minute, blend for 10 seconds, shake for 1 minute, blend for 10 seconds. The blended sample was warmed to room temperature before being opened and handled for analysis. 20 grams of each sample was then transferred to a 50 ml glass centrifuge bottle. Diethyl ether (25 ml) was added and the bottle was sealed and shaken using an orbital shaker at 200 rpm for 30 minutes. The extraction bottle was centrifuged at 1500×g for 10 minutes and the ether layer (supernatant) was recovered. The extraction was repeated (2×10 ml ether) and the pooled ether extract filtered over anhydrous sodium sufate to remove water. The ether was removed by evaporation under a gentle stream of nitrogen at 45° C. The percent oil yield was determined by weight. Extracted oil was stored in amber 40 ml vials at −20° C. until analyzed. The results are shown in Tables 2 and 3.

TABLE 2

| | | | | | | Brown |
|---|---|---|---|---|---|---|
| Oil used | FuFA | FuFA in | 3MND in | 3H-3MND | Off-flavor | sweet |
| to make | in oil | granola | granola | in granola | score | flavor |
| granola | (ppm) | (ppm) | (ppb) | (ppb) | (1-5⁺) | presence |
| Control | 0.91 | Not tested | ND | 2.34 | 1 | Yes |
| HOC oil | | | | | | |
| (untreated) | | | | | | |
| Commodity | 212 | 150 | 10.5 | 86.3 | 3 | No |
| soybean oil | | | | | | |
| (untreated) | | | | | | |
| HOSoy oil | 105 | 84 | 2.98 | 45.6 | 4 | No |
| (untreated) | | | | | | |
| #1* | | | | | | |
| HOSoy oil | 105 | ND | 19.2 | 42.9 | 4 | No |
| (untreated) | | | | | | |
| #2* | | | | | | |
| HOSoy oil | 70 | 49 | 1.73 | 28.6 | 1 | Yes⁺⁺ |
| (carbon- | | | | | | |
| treated) | | | | | | |

*same oil used to make 2 different granola samples
**ND = not detected
⁺Off-flavor scores: 1 = not detected/not different from control (HOC oil); 2 = can distinguish from control in side-by-side comparison; 3 = slight defect (off-flavor) detected without reference to control; 4 = significant perception of defect (off-flavor), flavor clearly deteriorated; 5 = unfit for end use
⁺⁺Brown sweet flavor was slightly less than control (HOC oil)

TABLE 3

| | 3MND in | 3MND in non-polar | 3MND in polar |
|---|---|---|---|
| Oil | oil (ppb) | carbon extract (ppb) | carbon extract (ppb) |
| HOSoy oil | <1 | 18.4 | 893 |

3MND was either very low (<1 ppb) or not detected in each of the oils tested, but 3MND was readily detectable in extracts from activated carbon, with the polar extract recovering a high concentration of 3MND (Table 3). 3H-3MND was not detected in carbon extracts. Based on the data in Tables 2 and 3, it was hypothesized that the reduction in FuFA levels in carbon-treated soybean oil relative to untreated soybean oil may have been due to conversion of FuFA to 3MND (and possibly 3H-3MND), which was then adsorbed by activated carbon. Interestingly, however, although activated carbon adsorbed 3MND, oil extracted from granola made with carbon-treated soybean oil still had a 3MND level (1.73 ppb) similar to granola made from untreated oil (2.98-19.2 ppb). Despite similar 3MND levels in granola made from untreated soybean oil and carbon-treated soybean oil, granola made from carbon-treated soybean oil tasted nearly identical to the control sample made from canola oil, including the presence of a brown, sweet flavor, while granola made from untreated soybean oil had a significant off-flavor. A similarly surprising observation was made with respect to 3H-3MND levels in granola made from treated (carbon-treated or heat-treated, as shown in Example 2) versus untreated oil, where 3H-3MND levels in granola did not predict off-flavor presence. Compare 3H-3MND levels shown in Table 2 and Table 5 versus the respective off-flavor scores.

### Example 2—Heat Treatment

HOSoy oil (Samples 10-12) and a blend of HOSoy and commodity soybean oil (75:25 blend; Sample 13) was heat-treated by maintaining the oil at various temperatures and times with agitation in the presence of oxygen to produce a heat-treated oil. All heat-treated oils were then deodorized using a standard oil deodorization method. Table 4 shows the conditions tested and whether off-flavor was detected in granola made with the deodorized, heat-treated oil.

TABLE 4

| | | Temperature | | | Off-flavor |
|---|---|---|---|---|---|
| Sample | Oil | (° C.) | Time | | detection |
| Control | RBD HOSoy oil | NA | NA | | Yes |
| (untreated) | | | | | |
| 10 | RBD HOSoy oil | 175-205 | 1 | hour | No |
| 11 | RBD HOSoy oil | 132 | 2-8 | hours | No |
| 12 | RB HOSoy oil | 90 | 2-8 | hours | No |
| 13 | HOSoy:Commodity | 132 | 4 | hours | No |

blend

As can be seen in Table 4, conditions that include a temperature of 90-205° C. (194-401° F.) and a time of 1 hour to 8 hours resulted in little to no off-flavor detected. Brown, sweet flavor was absent in all of the samples in Table 4.

To determine the effect of heat treatment on FuFA content, 3MND content, and 3H-3MND content, levels of these molecules were measured using gas chromatography-mass spectrophotometry (GC-MS) in untreated oil, deodorized, heat-treated oil (about 4 hours at 130-135° C.), and oil extracted from granola made using each oil. Granola was made and extracts were obtained using the methods described in Example 1. The results are shown in Table 5.

TABLE 5

| Oil used | FuFA | FuFA in | 3MND in | 3H-3MND | Off-flavor | Brown |
|---|---|---|---|---|---|---|
| to make | in oil | granola | granola | in granola | score | sweet |
| granola | (ppm) | (ppm) | (ppb) | (ppb) | (1-5) | presence |
| Control | 0.91 | Not tested | ND | 2.34 | 1 | Yes |
| HOC oil | | | | | | |
| (untreated) | | | | | | |
| Commodity | 212 | 150 | 10.5 | 86.3 | 3 | No |
| soybean oil | | | | | | |
| (untreated) | | | | | | |
| HOSoy oil | 168 | 122 | 3.57 | 59.8 | 2 | No |
| (untreated) | | | | | | |
| HOSoy oil | 57 | ND | ND | 62.5 | 1 | No |
| (heat- | | | | | | |
| treated) | | | | | | |

Based on the data in Tables 5, it was hypothesized that the reduction in FuFA levels in heat-treated soybean oil relative to untreated soybean oil may have been due to oxidative breakdown of FuFA. It was initially hypothesized that a reduced FuFA content would lead to improved flavor by preventing formation of 3MND and/or 3H-3MND in food made with a heat-treated food. However, while oil extracted from granola made with heat-treated soybean oil had no detectable 3MND, it still had a 3H-3MND level (62.5 ppb) similar to granola made from untreated oil (59.8 ppb). Yet, despite similar 3H-3MND levels in granola made from untreated soybean oil and heat-treated soybean oil, granola made from heat-treated soybean oil tasted nearly identical to the control sample made from canola oil (with the exception of the lack of brown, sweet flavor) while granola made from untreated soybean oil had a significant off-flavor.

Interestingly, deodorized, heat-treated HOSoy oil that was subsequently carbon-treated had a stronger brown, sweet flavor than granola made with deodorized, heat-treated HOSoy oil that was not carbon-treated.

The implementations described above and other implementations are within the scope of the following claims. One skilled in the art will appreciate that the present disclosure can be practiced with embodiments other than those disclosed. The disclosed embodiments are presented for purposes of illustration and not limitation.

1. A method of making a carbon-treated soybean oil, comprising:
a. providing a soybean oil;
b. treating the soybean oil under conditions that promote conversion of furan fatty acids to 3MND in the presence of an amount of activated carbon to produce a carbon-treated soybean oil; and
c. collecting the carbon-treated soybean oil, wherein the carbon-treated soybean oil has a reduced furan fatty acids content relative to the soybean oil.

2. The method of claim 1, wherein, the conditions comprise maintaining the soybean oil at a temperature of about 90° F. (32.2° C.) to about 120° F. (48.9° C.) in the presence of oxygen.

3. The method of claim 2, wherein the conditions comprise oil movement.

4. The method of claim 1, wherein the amount of activated carbon is at least 6% by weight of the soybean oil.

5. The method of claim 1, wherein the carbon-treated soybean oil has a furan fatty acids content is less than 100 mg/kg.

6-7. (canceled)

8. A carbon-treated soybean oil having a furan fatty acids content of less than 100 mg/kg.

9-10. (canceled)

11. A food comprising the carbon-treated soybean oil of claim 8.

12. A method of making a deodorized, heat-treated soybean oil, comprising:
a. providing a soybean oil;
b. maintaining the soybean oil at a temperature of 170° F. (76.7° C.) to 460° F. (237.8° C.) in the presence of oxygen for at least 30 minutes to make a heat-treated soybean oil;
c. deodorizing the heat-treated soybean oil to produce a deodorized, heat-treated soybean oil; and
d. collecting the deodorized, heat-treated soybean oil, wherein the deodorized, heat-treated soybean oil has a reduced furan fatty acid content relative to the soybean oil.

13. The method of claim 12, wherein the maintaining step is performed at a temperature of 260° F. (126.7° C.) to 300° F. (148.9° C.) for about 1 hour to about 6 hours.

14. (canceled)

15. The method of claim 12, wherein the maintaining step is performed in the presence of a bleaching clay.

16. The method of claim 12, wherein the maintaining step is performed in the presence of activated carbon.

17. The method of claim 12, further comprising:
e. treating the deodorized, heat-treated soybean oil under conditions that promote conversion of furan fatty acids to 3MND in the presence of an amount of activated carbon to produce a carbon-treated soybean oil; and
f. collecting the carbon-treated soybean oil.

18. The method of claim 17, wherein the conditions comprise maintaining the deodorized, heat-treated soybean oil at a temperature of about 90° F. (32.2° C.) to about 120° F. (48.9° C.) in the presence of oxygen.

19. The method of claim 18, wherein the conditions comprise oil movement.

20. The method of claim 17, wherein the amount of activated carbon is at least 6% by weight of the deodorized, heat-treated soybean oil.

21. A method of making a grain-based food product, comprising
a. combining ingredients to form an uncooked composition, the uncooked composition including:
i. a soybean oil having a furan fatty acids content of at least 100 mg/kg, and
ii. substantially no reducing sugar and a pH of less than 6; and
cooking the uncooked composition to produce the grain-based food product; or
b. providing a grain-based food piece; and
applying a coating to a surface of the grain-based food piece to make the grain-based food product, the coating including:
i. a soybean oil having a furan fatty acids content of at least 100 mg/kg, and
ii. no reducing sugar and a pH of less than 6.

22-24. (canceled)

25. A method of reducing furan fatty acid content in an oil, comprising:
a. providing an untreated oil having a furan fatty acid content of at least 100 mg/kg;
b. carbon-treating and/or heat-treating the untreated oil to produce a treated oil, where:
1) carbon-treating the oil comprises treating the oil under conditions that promote conversion of furan fatty acids to 3MND in the presence of an amount of activated carbon; and
2) heat-treating the oil comprises maintaining the oil at a temperature of 170° F. (76.7° C.) to 460° F. (237.8° C.) in the presence of oxygen for at least 30 minutes; and
c. collecting the treated oil, wherein the treated oil has a reduced furan fatty acids content relative to the untreated oil.

26. The method of claim 25, wherein the treated oil has a furan fatty acid content that is reduced by at least 15% relative to the untreated oil.

27. The method of claim 25, wherein the treated oil has a fatty acid content that is less than 100 mg/kg.
